(19) United States
(12) Patent Application Publication
(10) Pub. No.: US 2021/0022889 A1
(43) Pub. Date: Jan. 28, 2021

(54) BODY MOUNTED STABILIZING PROSTHESIS WITH ENVIRONMENTAL AWARENESS

(71) Applicant: Michael Isakov, Brooklyn, NY (US)

(72) Inventor: Michael Isakov, Brooklyn, NY (US)

(21) Appl. No.: 16/937,517

(22) Filed: Jul. 23, 2020

Related U.S. Application Data
(60) Provisional application No. 62/877,803, filed on Jul. 23, 2019.

(51) Int. Cl. A61F 2/70; A61F 2/54

(52) U.S. Cl. CPC A61F 2/70; A61F 2002/5038; A61F 2002/704; A61F 2/54

(57) ABSTRACT

The present invention is a robotic prosthesis comprising: a mounting plate designed to attach to a wearer; a first joint attached to the mounting plate, wherein the first joint provides for rotation about a fixed number of axes; an arm having a first end and a second end, wherein the first joint is secured to the first end; an attachment, connected to the second end of the arm, wherein the attachment has at least one camera; and a computing device, wherein the computing device is able to collect data from the at least one joints to control the positioning of the attachment.

### CROSS-REFERENCE TO RELATED APPLICATIONS

This application is a continuation-in-part (and claims the benefit of priority under 35 USC 120) of U.S. application No. 62/877,803 filed Jul. 23, 2019. The disclosure of the prior applications is considered part of (and is incorporated by reference in) the disclosure of this application.

### BACKGROUND OF THE INVENTION

The present invention relates to prosthesis systems, and more particularly to a prosthesis system with stabilization and identification features.

Situational awareness refers to the perception of events and context in space and/or time. This generally involves awareness of what is transpiring within the immediate vicinity of the person perceiving events, as well as developing an understanding of how information, events, context, and actions will affect the goals or objectives of an actor, individual, or organization, both in the short and long term.

Typically, situational awareness involves a projection into the future of the status of an individual after some variable has changed, such as the passage of time, or the occurrence of an event. Situation and awareness is a key field of study in a number of industries, particularly those that involve rapidly changing operational environments and high stakes, such as aviation, military operations, and emergency services.

Battlefields in war time scenarios are wrought with uncertainty. Maintaining lethality while securing allies' lives is highly desired by soldiers, families, generals, and nations overall due to the fragility and high value of human life.

There are methods for disassociating soldiers from uncertain environments by decoupling them from the battlefield with the use of drones, however, risking the lives of soldiers is still mandatory to be effective in war time environments.

Given that the use of soldiers in the battlefield is a necessity and the risk of death or injury remains high, augmenting a soldier's capability in both a defense and offensive way that eliminates uncertainty from the battlefield is highly desired. Soldier augmentation is a challenging task and may come at the cost of other battlefield advantages.

Therefore, creating a lightweight, well balanced, safe, easily installed, controlled, and self-aware prosthesis that serves to augment a soldier's defense and offensive capabilities, thereby increasing persistence and survivability on the battlefield, without hindering performance in any way is highly desired.

Such an environmentally aware, interactive prosthesis equipped with non-lethal auxiliary tooling would also be applicable to a broader set of industries where alerting and hands on multitasking are required—including but not limited to film production, construction, and emergency response services.

### SUMMARY

Accordingly, it is an objective of the present invention is a robotic prosthesis comprising: a mounting plate designed to attach to a wearer; a first joint attached to the mounting plate, wherein the first joint provides for rotation about a fixed number of axes; an arm having a first end and a second end, wherein the first joint is secured to the first end; an attachment, connected to the second end of the arm, wherein the attachment has at least one camera; and a computing device, wherein the computing device is able to collect data from the at least one joints to control the positioning of the attachment.

Accordingly, it is an objective of the present invention is a method of operation of a robotic prosthesis, comprising: collecting data from a series of cameras and a plurality of sensors; processing the collected data to identify the position of an attachment and of at least one target within a field of vision of the series of cameras; determining a position of the attachment which places the at least one target within a line of sight; and adjusting at least one of a series of joints to reposition the attachment to the desired position.

Accordingly, it is an objective of the present invention is a robotic prosthesis comprising: a mounting plate designed to attach to a wearer; a series of joints, wherein each joint has one degree of rotation and at least one sensor to collect positioning data; a series of arms having a first end and second end, wherein one of the series of joints is attached to the first end and the second end of each of the series of arms; an attachment connected to the joint with an exposed end, and wherein the attachment has at least two cameras facing in opposite directions relative to the mounting plate position; and a computing device, wherein the computing device is able to collect data from the series of joints and the at least two cameras.

### BRIEF DESCRIPTION OF THE DRAWINGS

FIG. 1 depicts an isometric view of a mounted prothesis, in accordance with one embodiment of the present invention.

FIG. 2 depicts an isometric view of the mounted prothesis, in accordance with one embodiment of the present invention.

FIG. 3 depicts an exploded view of the mounted prothesis, in accordance with one embodiment of the present invention.

FIG. 4 depicts an isometric view of a mounted prothesis, in accordance with another embodiment of the present invention.

FIG. 5 depicts a block diagram of the logic of the mounted prothesis, in accordance with another embodiment of the present invention.

FIG. 6 depicts an image of a computing environment, in accordance with one embodiment of the present invention.

FIG. 7 depicts an image of the field of vision of the mounted prothesis, in accordance with the embodiment of the present invention.

FIG. 8 depicts an image of the field of vision of the mounted prothesis, in accordance with the embodiment of the present invention.

FIG. 9 depicts an exploded view of a joint, in accordance with the embodiment of the present invention.

FIG. 10 depicts an image of the joint assembly, in accordance with the embodiment of the present invention.

FIG. 11 depicts a flow chart of the operation of the mounted prothesis, in accordance with the embodiment of the present invention.

### DETAILED DESCRIPTION OF THE INVENTION

The present invention provides a device that is self-balancing and provides for situational awareness to identify and deal with potential hazards. The prosthesis consists of a dynamic robot arm-like assembly that protrudes from a wearer's back, has multiple degrees of freedom, maintains stability with onboard orientation sensors, is able to detect objects using an onboard computing device, camera, and peripheral sensors, and is able to orient itself to fire at a given target with its motors, environmental awareness software, and an onboard projectile shooting device. This device also has many uses in the civilian space. This device can be used in as a camera mount to free the wearers hands for various activities, and can also be used as a safety device for various jobs or activities that would require the wearer to be concerned about hazards in all directions with alternative auxiliary tooling at the end of the prosthesis.

The present invention seeks to provide a solution to the problem of uncertainty and survivability by providing a system to augment the lethality of a soldier on the battlefield through a multi-degree of freedom self-stabilizing robotic prosthesis assembly with cameras, sensors, and an interchangeable projectile mount and digitally controlled shooting system, and provide a system for protecting a person through a self-aware robotic arm that can identify and alert the person to threats. In another embodiment, the device can be used to free to wearers hands from needed to hold various pieces of equipment such as a camera, alert them of key environmental assets, enhance hands on multitasking ability, and also allow them to focus on a specific task at hand.

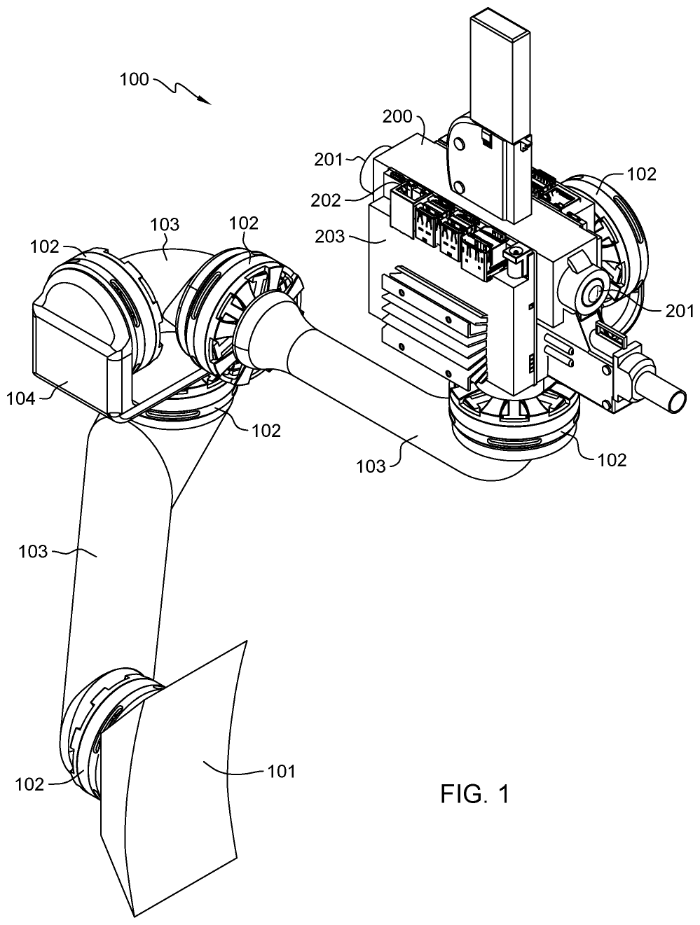

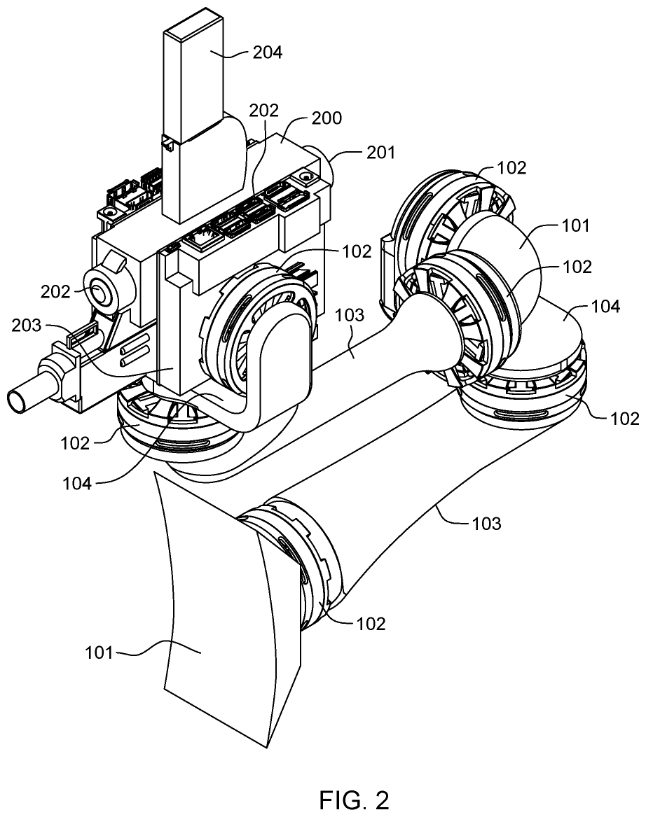

FIG. 1 depicts an embodiment of the should mounted prothesis 100, in accordance with one embodiment of the present invention. The shoulder mounted prothesis 100 (device) is comprised of a base plate 101, joints 102, arms 103, mounting plates 104, and an attachment 200.

The base plate 101 is designed and formed to secure to the wearer. This can be achieved through specialized attire or armor worn by the person that has attachment points for the base plate. In alternative embodiments, the base plate 101 is secured to a larger piece of attire or equipment worn by the person, or which is fitted around the person. The base plate 101 has an ergonomic design to assist in maintain the balance of the wearer based on the weight distribution of the device.

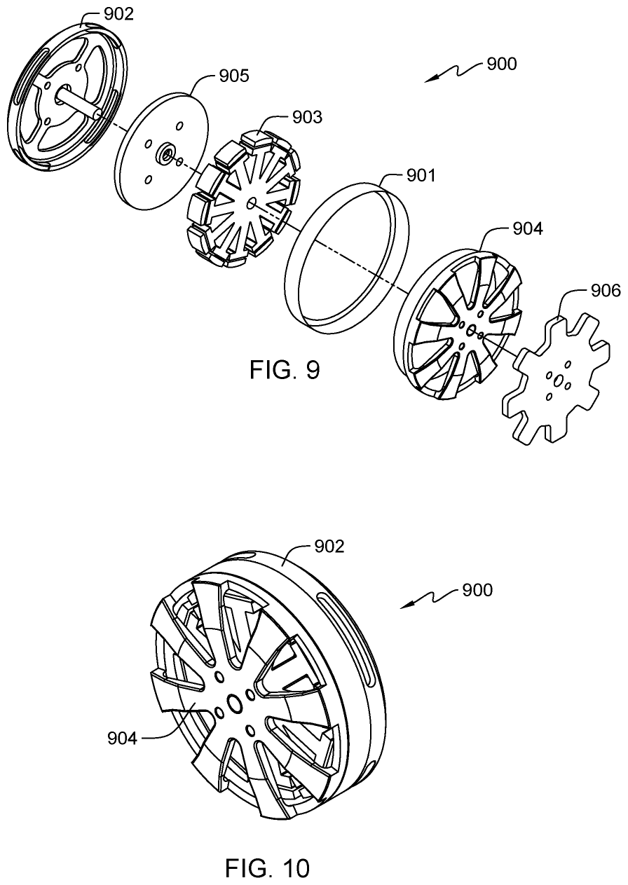

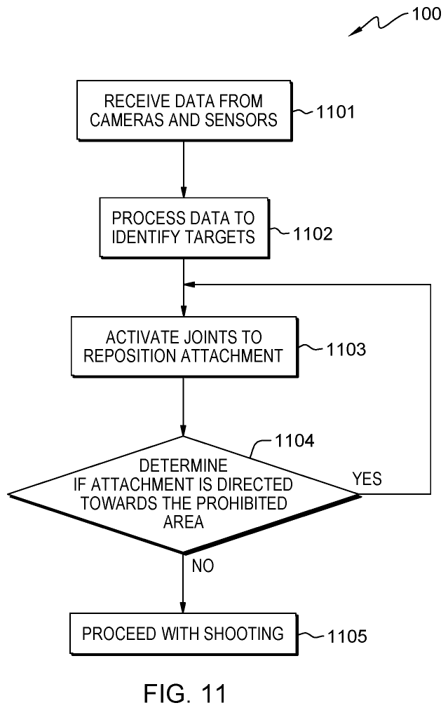

The base plate 101 is secured to a joint 102. The joints 102 provide for the ability to rotate about varying degrees (axes) of rotation. Based on the design and requirements based on the number of joint 102 and overall design and purpose of the device. The joint 102 includes a motor, actuator, and sensors 105 to both receive and send data, so that a central computing unit is able to control the joint 102. In one embodiment, each joint 102 is equipped with an accelerometer at the base section and aft section of the motor to provide data related to the positioning of each section of the motor. The sensors 105, various types of positioning sensors 105, accelerometers, gyro sensors 105, and the like which are used to determine the position of the joint 102. The joint 102 are designed to provide for rotation in one axis or one degree of freedom. Through the use of multiple joint 102, there is the ability for a full 360 degrees of positioning of the attachment 200. The joint 102 contain a brushless motor, accelerometers, and additional sensors 105. The brushless motor provides the power source to adjust the positioning of each joint 102, where the accelerometers and sensors 105 assist the controller in the necessary adjustment to each joint 102 to achieve the desired positioning and orientation of the attachment 200. The joint 102 are designed to provide unencumbered electrical communication of each joint 102 and the attachment. Depicted in FIGS. 9 and 10 are an embodiment, of the joint 900. Where the joint is comprised of a back plate 904, a front plate 902 which house the internal motor components 903 and 901. The front plate 902 and back plate 904 have integrated sensors 105 which assist the logic in determining the desired rotation (degree and direction) to position the attachment 200 in the ideal position and in the ideal direction. In the depicted embodiment sensors 905 and 906 are placed on each plate respectively to both collect data based on the positioning of the joint, the rotation of each plate, and use this information to assist the logic components to properly orientate the attachment 200. The sensors 905 and 906 may be various sizes and types based on the overall joint 900 design. The sensors may contain accelerometers or other integrated sensors on a circuit board.

The motor permits the joint 102 to rotate through the degree(s) of movement based on the joint design. within the joint 102 consists of an axial brushless motor having a base section and an aft section, wherein the base section contained the stator assemblies and the coils, with a central shaft. In the depicted embodiment each joint 102 is able to rotate about one axis, through the use of six (6) joints 102, the attachment is able to be directed in a full 360 degree of positioning.

Secured to the joint 102 is an arm 103. The arm 103 is of a predetermined shape, size, length, and bends to assist in positioning the attachment 200. In an embodiment, the attachment 200 is designed to be positioned to the side of the user's head. Through the design of the arms 103 the attachment is able to accomplish this position as well as a stored position. The arms 103 is made of a light weight material capable of supporting the weight of the weapon or attached piece of equipment, and designed in conjunction with the other arms 103 to provide a near 360 degree of direction for the weapon, camera, or device attached. In some embodiments, the arms 103 have embedded sensors 105 to provide additional information which may be absent from the data collected from the joint 102. The arms 103 may be made from various plastics, metals (titanium, aluminum, etc.) or composite materials. In the depicted embodiment, the device is designed to position the attachment 200 on each side of the wearers head and slightly behind the wearer. This removes the device 100 from the wearers field of vision.

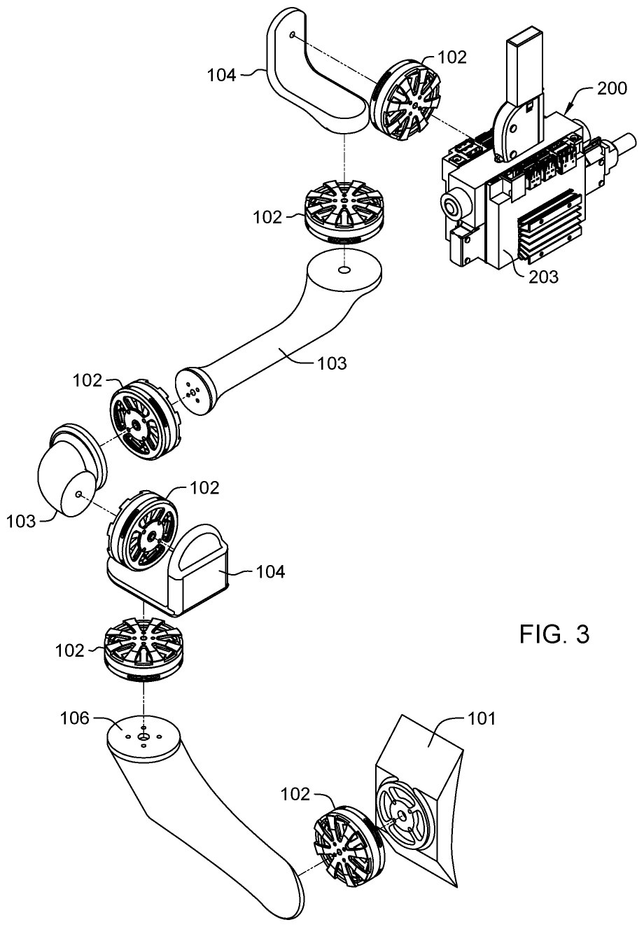

The mounting plates 104 provides for the between joints. In the depicted embodiment, the attachment 200 (e.g. weapon, camera, or equipment) is secured to a joint 102 which is attached to the mounting plate 104 to provide an additional degree of rotation. The attachment 200 may be directly attached to the mounting plate 104, or as shown in FIG. 3, the attachment 200 is secured directly to the joint 102. The mounting plate 104 may be made from various plastics, metals (titanium, aluminum, etc.) or composite materials.

The depicted embodiments show a setup where there are two arm 103 portions and six joint 102. Wherein the attachment 200 has an additional and integrated joint 102. In an additional embodiment, shown in FIG. 3, the device 100B having two arms 103 and three joints 102.

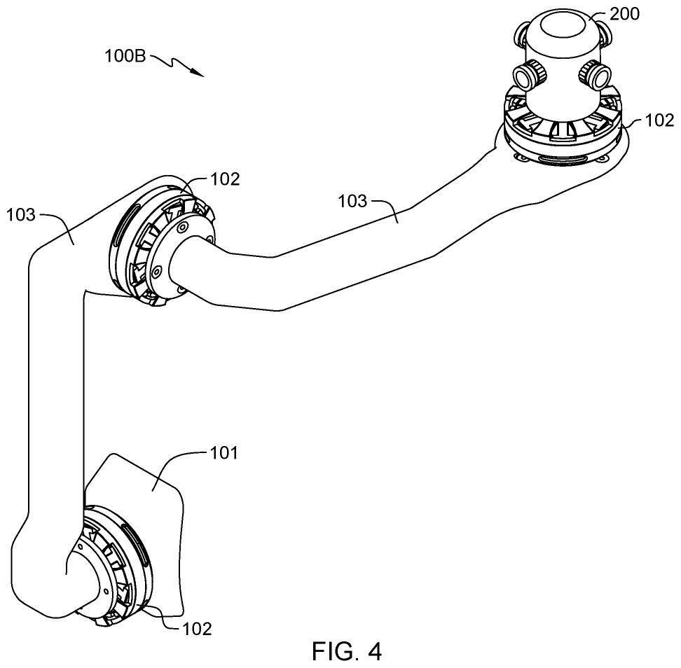

The attachment 200 is shown secured to the end of the device 100 and provides for a plurality of abilities. In the depicted embodiment, the attachment 200 is a weapon which has the ability to shoot projectiles at a target. The attachment 200 has a camera 201 on the front and rear surfaces of the attachment 200 to provide additional information for the computing device. The attachment 200 is equipped with a plurality of connectors 202. In the embodiment depicted in FIG. 3 the attachment 200 is shown to have multiple cameras 201 without the ability to shoot a projectile. In the exploded embodiment, each joint 102 and arm 103 is shown with attachment points 106. The joints 102 are secured to the arms 103. In the depicted embodiment, computing device 203 is attached to the attachment 200. The computing device 203 provides for the processing and collection of the data from the cameras 202, the sensors 105, and the other data collection components of the device 100. In some embodiments, the attachment 200 may have more than one computing device 203 which is secured to the exterior of the attachment 200. In some embodiments, the computing device 203 is contained within the attachment (see FIG. 4) or integrated into the back plate 101 or within one or more of the arms 103. In the depicted embodiment, the attachment 200 has ammunition containment 204. Based on the intended purpose of the device 100, the attachment 200 may be fitted with a variety of abilities, connectors, or setups provided the attachment 200 has at least one camera 201. This may include recording via cameras, weapons to provide varying degrees of offensive and defensive nature, or robotic arms, laser systems, and other auxiliary tooling. The attachment 200 housing is equipped with a series of integrated cameras 201, both forward facing and rear facing. The attachment 200 may have an integrated computing system. The attachment 200 may have additional attachments such as weapons, or other dedicated tools.

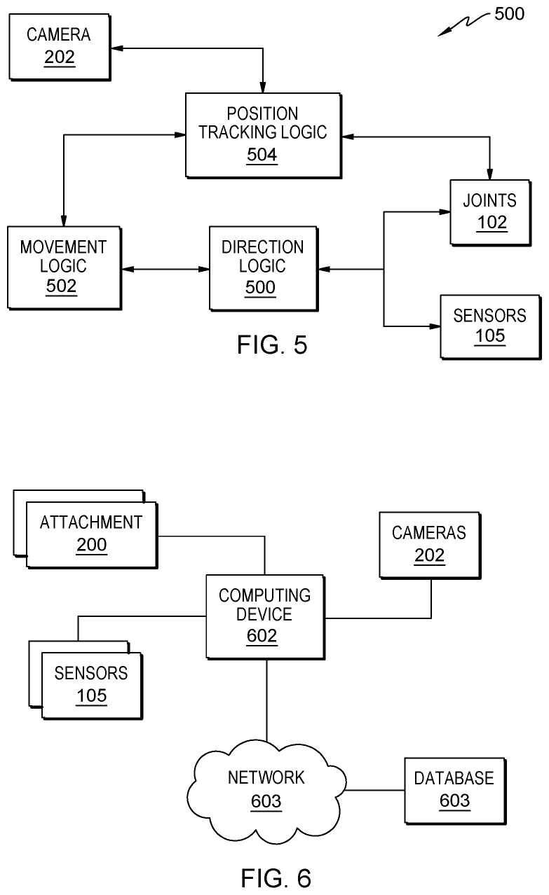

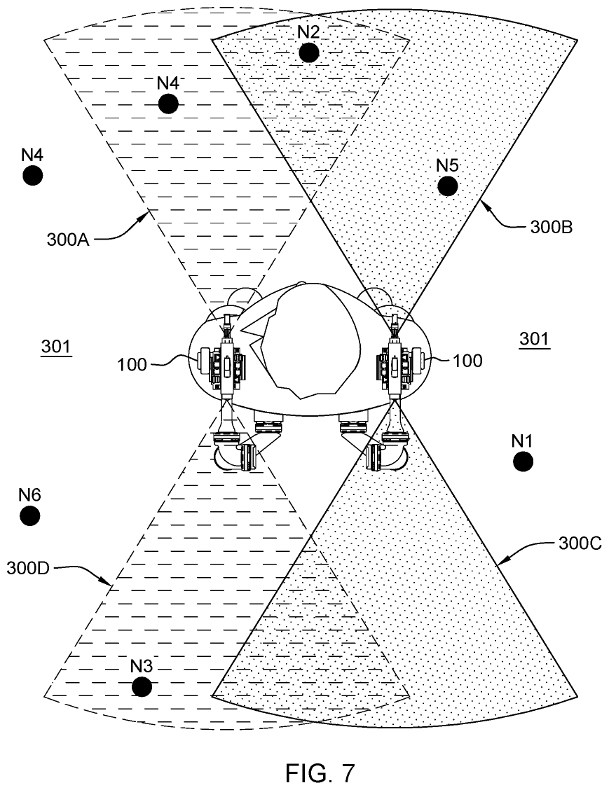

FIG. 5 is a schematic representation of a control system configured to maintain the balance, positioning, and identification of threats or items of interest of the attachment 200, according to an exemplary embodiment. Other control components shown in FIG. 6 that are outside of the control system are discussed with respect to FIG. 7, below. The control system comprises the motors within the joint 102, the sensors 105 within the joint 102, the direction logic 501, the movement logic 502, and the position tracking logic 504. In operation, the direction logic 501 receives a balance signal from the sensor 105 and controls the motors, for instance with a control signal, to redirect the positioning of the attachment 200.

The sensors 105 are disposed within the joint 102, arms 103, mounting plate 104, base plate 101, and the attachment 200, in various embodiments. The sensor 105 can comprise, for example, a measurement system configured to measure acceleration along the axes of the joint 102. Accordingly, the sensor 105 can comprise a set of accelerometers and/or gyroscopes, for example. The direction logic 501 uses the acceleration and angular measurements along the axes, in particular, to determine the relative direction of the attachment 200. It will be appreciated that this positioning constitutes changing the base angle from a target base angle. This target base angle is the base angle at which the system is estimated to be balanced. Based on this determination, the direction logic 501 determines whether to move the joint 102 to counteract the movement of the wearer, and the movement of the target. The change in the orientation of the attachment 200 as the direction logic 501 controls the motors in the joint 102 is then detected by the balance sensor 105 to close a feedback loop.

Movement of the attachment 200 can comprise rotating of the joint 102 and the attachment 200 to point the attachment in a particular direction or at a particular location while maintaining the balance of the attachment 200 through the wearer's movements. A position tracking logic 504 configured to track the location and orientation of the attachment 200 and the location or a target relative to either the internal or external frame of reference using the onboard camera systems and computing device to translate visual data to a set of movements based on the task assigned to the prosthesis. In some embodiments, the position tracking logic 504 tracks other information by monitoring the rotation of the joint 102 and/or by monitoring other sources like the sensor 105.

The position tracking logic 504 can track the location and the orientation of the attachment 200 and the targets through the use of the cameras 202, which will feed video input into a computer vision model in real time enabling the processing and identifying of targets and relative positions to prioritize. Through the known location of targets, the joint 102, and the arm 103 designs, the position tracking logic 504 is able to determine the position and direction of the attachment 200, as well as the future position and directions of the attachment at any moment in time. Location and orientation can also be tracked through the use of range finding equipment such as sonar, radar, and laser-based systems, for instance. Such equipment can be either be part of the device or external thereto. In the latter case, location and orientation information can be received by the position tracking logic 504 through a wireless communication link. Devices or logic for monitoring wheel rotation, as well as the range finding equipment noted above, comprise examples of position sensors 105.

Movement logic 502 is configured to receive at least the location information from the position tracking logic 504. The movement logic 502 can compare the received location information against a target location which can be any point within the relevant frame of reference. If the location information received from the position tracking logic 504 is different than the target location, the movement logic 502 directs the direction logic 501 to point the attachment to the target location. Where the target location is fixed, the movement logic 502 keeps the attachment fixed on the target and counteracts the movement of the wearer.

For the purposes of moving/redirecting the attachment 200 to a new location, the direction logic 501 has the additional capability to change the focus point of the attachment 200 to initiate a redirection of the sensors 105 and motors to the new focus point. Then, having established the new focus point, the direction logic 501 controls the motor to adjust accordingly to move the joint 102 and arms 103 to the new focus point, while also maintaining a stable relative position to the wearer. With the center of gravity of the attachment 200, the direction logic 501 maintains a center of gravity ideal for the performance of the attachment 200 and the wearer.

In some embodiments, the movement logic 502 can also compare orientation information received from the position tracking logic 504 against a target orientation. If there is a difference between the two, the movement logic 502 can instruct the direction logic 501 to reposition the attachment 200 to the target orientation.

Target locations and orientations can be determined by the movement logic 502 in a variety of ways. In some embodiments, the movement logic 502 can be programmed to execute moves at particular times or in response to particular signals. In other embodiments, the attachment 200 is configured to act autonomously, and in these embodiments the attachment comprises autonomous logic configured to update the movement logic 502 as needed with new targets. The movement logic 502 can also be configured, in some embodiments, to receive location and orientation targets from a human interface.

In some embodiments, the device 100 also comprises a control input logic configured to receive movement control signals from a movement control input device. Control input logic may be further configured to calculate a target location based on these signals, and to communicate the target location of the movement logic 502. Movement control input device may comprise a joystick, mouse, headset, position sensor, processor, or some other device configured for a user to indicate a target location or movement through the human interface which is remotely controlled.

A first position can be used as a resting state when not in use. A static idling mode may persist until the cameras detect a key asset or target upon which movement commands, alerting the wearer, or other attachment triggers are completed. The first position may then be returned to when the prosthesis has finished responding to the signal.

Shows a block diagram representation of a robotic system for operating the device according to an embodiment of the present invention. The system comprises the multi-armed device, a controller, such as a processor. The multi-armed device is in direct communication with the controller or can be in communication across a network. The network can comprise an Ethernet, a local area network, a wide area network, the Internet, or the like. The tracking sensor can each be in direct communication with the controller, as shown, or in communication through a network.

The device is not limited to a weapon, but can include any robot, whether mobile or fixed at a location, that includes a plurality of cameras. In some embodiments, such as those embodiments in which the device is characterized by a generally mounted device. Embodiments of the device can include sensors 105 which are devices that are configured to provide data from which the spatial location and aim of the cameras relative to a frame of reference can be calculated. Accordingly, sensors 105 can provide data concerning the angles of the joint 102 of the device 100. Other sensors 105 can be configured to measure other characteristics of the device such as rotational attributes like the amount of, or the rate of change of, pitch, roll and yaw. sensors 105 can comprise, in various embodiments, an inertial measurement unit (IMU), optical encoders and/or linear potentiometers. The device may also include actuators like robotic hands or other tooling for specific use cases.

The device can optionally include logic for performing video encoding and compression of the signals produced by the cameras. The device can optionally also include one or more data sources. Examples of data sources include such devices as microphones, a global positioning receiver, and environmental sensors 105 such as for temperature, atmospheric sampling, radiation monitoring, etc.

The cameras also comprise at least one camera configured to aim in a direction different than that of the other cameras, but with a field of view that at least partially overlaps the field of view of at least one of the cameras. It will be appreciated that some embodiments include sufficient cameras such that their overlapping fields of view of the set of cameras encompass an entire 360° field of view. One or more of the cameras can include, for example, an omni-lens, a fisheye lens, or a telephoto lens.

As noted above, a field of view of a camera at least partially overlaps the field of view of at least one of the cameras. It should be noted, however, that not every camera is required to have a field of view that overlaps with a field of view of a camera, so long as at least one camera has a field of view that overlaps in this manner. Although not illustrated, in various embodiments cameras can be aimed in the direction opposite to the direction that of the pair of cameras is aimed, as well as at arbitrary angles to the direction that of the pair of cameras is aimed, such as 30°, 45°, 60°, 90, 120°, 135°, and 150°.

The cameras can be mounted internally to the device, external to the device, or some can be mounted internally while some are mounted externally. It should be understood that the cameras need not be mounted on the device, and in some embodiments the cameras are attached to the device in other ways, such as by being supported on a boom extending from the device. It will also be appreciated that the cameras do not need to all be fixed to the same part of the device. For instance, some cameras can be attached to the device, while another camera can be attached to the arms 103 or joint 102 or even to the wearer.

In order to simplify the process of stitching together the various video signals received from the several cameras to produce a composite video signal from which an image can be rendered, the cameras are optionally arranged around a common reference point. In other words, a centerline of the common field of view should intersect the centerlines of the fields of view of the cameras.

The logic of the controller also can include lens correction logic. The lens correction logic is configured to receive the decompressed video signals from the decompression logic and remove image distortions such as those distortions due to lens curvature to produce corrected video signals from each of the cameras. The corrected video signals are then provided to image rendering logic of the controller, discussed below. In those embodiments in which the controller does not include lens correction logic, video decompression logic provides decompressed uncorrected video signals to the image rendering logic.

Figure shows a representation of a robot view comprising a view corresponding to the field of view of the video cameras and peripheral views corresponding to the fields of view of cameras according to an embodiment of the present invention. The view corresponds to the field of view of the video cameras as if projected onto a screen at some distance in robot space. Likewise, each peripheral view corresponds to the field of view of a peripheral camera. The view can also comprise other views omitted from for clarity, such as the views corresponding to the individual fields of view of the individual cameras.

In some instances, the device may be pointed at or obstructed by the wearer, the controller through the logic determines that the wearer is within the field of vision of the device and an alert is sent to the controller to redirect the device. A fire control subsystem is locked out and the controller redirects the device. For all embodiments, the controller is calibrated to not damage or interfere with the wearer based on mounting points and geometrically defined relative positions.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods, and computer program products according to various embodiments of the present invention. In this regard, each block in the flowcharts may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the flowchart illustrations, and combinations of blocks in the flowchart illustrations, can be implemented by special purpose hardware-based systems that perform the specified functions or acts or carry out combinations of special purpose hardware and computer instructions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Network 603 may be a local area network (LAN), a wide area network (WAN) such as the Internet, any combination thereof, or any combination of connections and protocols that can support communications between computing device 602 in accordance with embodiments of the invention. Network 603 may include wired, wireless, or fiber optic connections.

Computing device 602 may be a management server, a web server, or any other electronic device or computing system capable of processing program instructions and receiving and sending data. In some embodiments, computing device 602 may be a laptop computer, tablet computer, netbook computer, personal computer (PC), a desktop computer, or any programmable electronic device capable of communicating with cameras 201, attachment 200, and sensors 105 via network 102. In other embodiments, computing device 602 may represent a server computing system utilizing multiple computers as a server system, such as in a cloud computing environment. In another embodiment, computing device 602 represents a computing system utilizing clustered computers and components to act as a single pool of seamless resources. Computing device 602 may be integrated into the attachment 200 or may be a separate entity connected to the attachment 200. In some embodiments, the system is driven by an onboard computing device that processes the image, video, and sensor data, processes that data locally or through a network 602, and responds to inputs by controlling the actuators to reorient the assembly and fire at targets.

Database 604 may be a repository that may be written to and/or read by computing device 602. Information gathered from sensors 105, cameras 201, and other data collecting components and devices may be stored to database 604. Such information may include previous scores, audio files, textual breakdowns, facts, events, and contact information. In one embodiment, database 604 is a database management system (DBMS) used to allow the definition, creation, querying, update, and administration of a database(s). In the depicted embodiment, database 604 is connected to network 603.

The system interfaces with external computing devices that are local to the wearer or via the network 603 with the onboard computing device which is configurable from third party apps. The system receives software updates and is able to stream video, audio, sensor data, and a control protocol over a wireless connection. The cameras 201 and sensors 105 are connected to a computing device 602, which is also connected to the actuators which control the position and direction of the cameras 201 and sensors 105. The computing device is connected to a network. Typically, the connection is wirelessly, but can be wired. In some embodiments, the cameras 201 and the sensors 105 are various weapons and/or tools which are attached to the arms 103.

The system employs methods through its onboard computing device to process input data from the environment ranging from video, audio, motion, and positional data to build a digital model of its surroundings (step 1101). The computing device may receive data from the sensors within the joints, arms, or attachments to determine the present position of each component, and determine which components need to be activated or adjusted to achieve a desired positioning. Using customizable computer vision models the system is able to recognize objects in its surrounding environment and then store their locations (step 1102). In some embodiments, this step may also determine the positioning of the attachment as well as the positioning of the joints and arms, through the collected data from the sensors. On command or autonomously, the system may employ several methods to lock onto and/or interact with the surrounding targets (step 1103). This may be accomplished to adjusting one or more of the joints, to achieve the desired positioning of the attachment For each target, metadata is collected and calculated using algorithmic techniques and sensor data to identify such things as object type, object distance, object priority, object threat level, object motion, object rates of motion, object orientation, object behavior, and other object metadata. The system is able to determine if the user/wearer is within the field of vision or line of fire of the attachment 200, and if so, the logic and computing device is able to reorientation the attachment 200 so that the wearer/user is out of the field of vision or line of fire of the attachment 200, or properly reposition itself so that the wearer is out of view or outside the line of fire (step 1104). In some embodiments, the device 10 is calibrated based on the wearer, to determine the prohibited area, and when in operation the device 100 does not permit the attachment to enter or be pointed into or across this space.

FIG. 7 shows the environmental awareness method for object detection. In the depicted embodiment, there are two forward facing cameras 201 and two rear facing cameras 201 attached to the arms 103, and the field of vision 300A, 300B, 300C, 300D of the cameras 201 overlap in front of and behind the user. Additionally, the space not visible to the camera 301 is shown as empty space. The device may scan these areas to assist in detecting threats outside of the field of visions. Based on the objects detected, the computing device is able to prioritize the targets based on various characteristics. For example, which target is a potential threat to the user (e.g. N1, N2, N3, and N5). In the depicted image, there are various targets outside the viewable area of the cameras. The head and limbs of the wearer are defined as no-go zones which the controller considers when prioritizing movements to point the device in a specific direction.

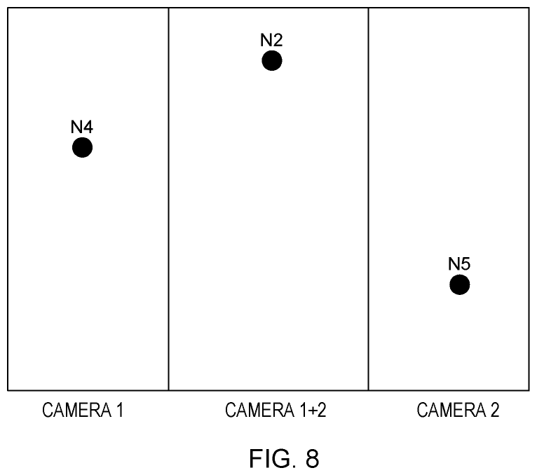

FIG. 8 shows the viewable space of the cameras and the section that is covered by both cameras 201 and is able to detect the location of the target within the field of vision. The cameras 201 are able to track the targets throughout the field. The field of vision is a two-dimension or three-dimensional space based on the sensor arrays equipped on the prosthesis. In the depicted embodiment, the targets N4 N2, and N5 are positioned based on their position relative to a two-dimensional space. In additional embodiments, the targets N4. N2, and N5 could be positioned based on a three-dimensional space, wherein N5 would be the closet to the user, and N2 would be the farthest from the user. In some embodiments, the cameras 202 are able to track a target, or sense targets outside of the viewable area and the arm 103 is able to move the camera 202 so the sensed target becomes within the viewable area. The positioning of the targets within the viewable space shows the proximately to the user. This may be accomplished through sensors which are able to detect noise, movement, or the like.

In some embodiments, the cameras 202 have 360-degree coverage. In some embodiments, the device is able to remember locations of targets which are identified and reference these locations in the future. In some embodiments, the device is able to track not only position but also velocity based on fine time interval frame-by-frame differences in camera footage as well as the array of onboard sensors.

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein that are believed as maybe being new, this understanding, as indicated by use of the term “present invention,” is tentative and provisional and subject to change over the course of patent prosecution as relevant information is developed and as the claims are potentially amended.

The foregoing descriptions of various embodiments have been presented only for purposes of illustration and description. They are not intended to be exhaustive or to limit the present invention to the forms disclosed. Accordingly, many modifications and variations of the present invention are possible in light of the above teachings will be apparent to practitioners skilled in the art. Additionally, the above disclosure is not intended to limit the present invention. In the specification and claims the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g. attached, adhered, joined) are to be construed broadly and may include intermediate members between a connection of elements and relative movement between elements. As such, joinder references do not necessarily infer that two elements are directly connected and in fixed relation to each other. Moreover, network connection references are to be construed broadly and may include intermediate members or devices between network connections of elements. As such, network connection references do not necessarily infer that two elements are in direct communication with each other. In some instances, in methodologies directly or indirectly set forth herein, various steps and operations are described in one possible order of operation, but those skilled in the art will recognize that steps and operations may be rearranged, replaced or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements and/or substantial equivalents.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

What is claimed is:

1. A robotic prosthesis comprising:
a mounting plate designed to attach to a wearer;
a first joint attached to the mounting plate, wherein the first joint provides for rotation about a fixed number of axes;
an arm having a first end and a second end, wherein the first joint is secured to the first end;
an attachment, connected to the second end of the arm, wherein the attachment has at least one camera; and
a computing device, wherein the computing device is able to collect data from the at least one joints to control the positioning of the attachment.

2. The robotic prosthesis of claim 1, further comprising, a second joint secured to the second end of the arm, wherein the second joint is able to rotate about a different axes as the first joint.

3. The robotic prosthesis of claim 1, wherein the joints are comprised of a first half, a second half, and an integrated motor, wherein a sensor is connected to the first half and another sensor is connected to the second half, and both sensors are able to transmit data to the computing device.

4. The robotic prosthesis of claim 1, wherein the computing device is integrated into the attachment.

5. The robotic prosthesis of claim 1, wherein the computing device is integrated into the mounting plate.

6. The robotic prosthesis of claim 1, wherein the attachment has a camera facing in a first direction and a second camera facing in an opposite direction.

7. The robotic prosthesis of claim 1, wherein the attachment has a plurality of cameras viewing substantially 360 degrees.

8. A method of operation of a robotic prosthesis, comprising:
collecting data from a series of cameras and a plurality of sensors;
processing the collected data to identify the position of an attachment and of at least one target within a field of vision of the series of cameras;
determining a position of the attachment which places the at least one target within a line of sight; and
adjusting at least one of a series of joints to reposition the attachment to the desired position.

9. The method of operation of a robotic prosthesis of claim 8 further comprising, calibrating the attachment to avoid a predetermined space around the user.

10. The method of operation of a robotic prosthesis of claim 9 further comprising, readjusting the series of joints based on the calculation that the attachment line of sight would be within the predetermined space around the user.

11. The method of operation of a robotic prosthesis of claim 8 further comprising, recalibrating the series of joints based on the movement of the user to maintain the attachment in a predetermined position.

12. The method of operation of a robotic prosthesis of claim 8 further comprising, collecting data from a set of sensors within each of the series of joints, wherein the set of sensors produce data relative to one another.

13. The method of operation of a robotic prosthesis of claim 8 further comprising, calibrating, the attachment based on the number of joints and a set of properties associated with a series of arms.

14. The method of operation of a robotic prosthesis of claim 8 further comprising, determining a change in the orientation of the attachment; and
determining an adjusting to a group of the series of joints based on the change in orientation of the attachment.

15. The method of operation of a robotic prosthesis of claim 8 further comprising, processing the record video from the series of cameras and stitching the recorded video together and correcting the video feed.

16. A robotic prosthesis comprising:
a mounting plate designed to attach to a wearer;
a series of joints, wherein each joint has one degree of rotation and at least one sensor to collect positioning data;
a series of arms having a first end and second end, wherein one of the series of joints is attached to the first end and the second end of each of the series of arms;
an attachment connected to the joint with an exposed end, and wherein the attachment has at least two cameras facing in opposite directions relative to the mounting plate position; and
a computing device, wherein the computing device is able to collect data from the series of joints and the at least two cameras.

17. The robotic prosthesis of claim 16 further comprising, position tracking logic configured to correct the movement of the series of joints based on movement of a target and the movement of the user.

18. The robotic prosthesis of claim 16 further comprising, direction logic configured to adjust the series of joints to maintain the attachment's field of sight on a predetermined target.

19. The robotic prosthesis of claim 16 further comprising, movement logic configured to adjust specific joints based a fixed or adjusting positioning of the attachment.

20. The robotic prosthesis of claim 16 further comprising, video compression logic, configured to combine video signals to create a meshed video signal.
